(19) United States
(12) Patent Application Publication
(10) Pub. No.: US 2016/0168089 A1
(43) Pub. Date: Jun. 16, 2016

(54) PROCESS FOR THE MANUFACTURE OF IDALOPIRDINE

(71) Applicant: H. Lundbeck A/S, Valby (DK)

(72) Inventors: Mikkel Fog JACOBSEN, Frederiksberg (DK); Ole NIELSEN, Valby (DK)

(21) Appl. No.: 14/965,308

(22) Filed: Dec. 10, 2015

(30) Foreign Application Priority Data
Dec. 12, 2014 (DK) PA 2014 00721

(51) Int. Cl. C07D 209/14; C07D 209/08

(52) U.S. Cl. CPC C07D 209/14; C07D 209/08

(57) ABSTRACT

Disclosed herein is a process for the preparation of idalopirdine and pharmaceutically acceptable salts thereof.

### FIELD OF THE INVENTION

The present invention relates to the preparation of N-(2-(6-fluoro-1H-indol-3-yl)-ethyl)-3-(2,2,3,3-tetrafluoropropoxy)-benzylamine, INN-name idalopirdine, and pharmaceutically acceptable salts thereof.

### BACKGROUND

N-(2-(6-fluoro-1H-indol-3-yl)-ethyl)-3-(2,2,3,3-tetrafluoropropoxy)-benzylamine is a potent and selective 5-HT₆ receptor antagonist which is currently in clinical development. Its chemical structure is depicted below as Compound (I).

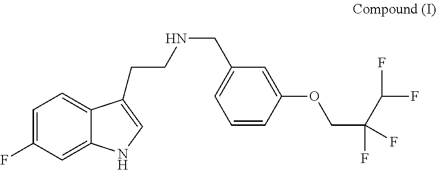 [SMILES: Fc1ccc2c(CCNCc3cccc(OCC(F)(F)C(F)F)c3)c[nH]c2c1]

The synthesis of N-(2-(6-fluoro-1H-indol-3-yl)-ethyl-(2,2,3,3-tetrafluoropropoxy)-benzylamine, its use for the treatment of disorders such as cognitive dysfunction disorders, and pharmaceutical compositions comprising this substance are disclosed in U.S. Pat. No. 7,157,488 (“the '488 patent”). The '488 patent further describes the preparation of the corresponding monohydrochloride salt.

Although the synthetic methods disclosed in the above-identified reference suffices to prepare small quantities of material, it suffers from a variety of safety issues, low yields or processes that are not amendable to large scale synthesis.

A method of manufacture useful for the production of kilogram quantities of material for preclinical, clinical and commercial use is disclosed in international patent application No. WO2011/076212.

The method of manufacture as disclosed in WO2011/076212 starts from commercially available 6-fluoroindole and is outlined in Scheme A.

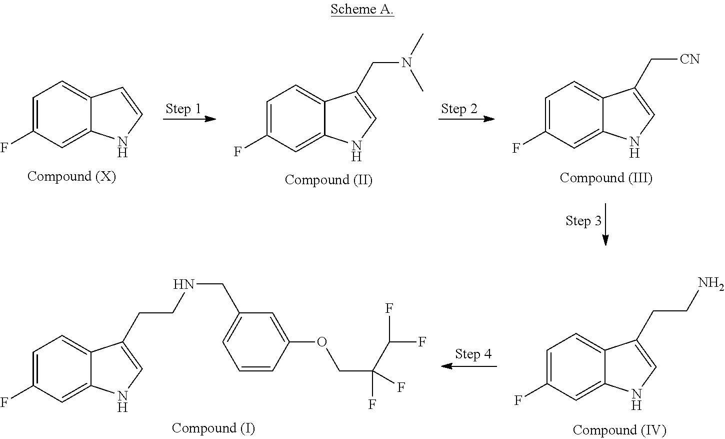 [SMILES: Fc1ccc2cc[nH]c2c1 ; CN(C)Cc1c[nH]c2cc(F)ccc12 ; N#CCc1c[nH]c2cc(F)ccc12 ; Fc1ccc2c(CCNCc3cccc(OCC(F)(F)C(F)F)c3)c[nH]c2c1 ; NCCc1c[nH]c2cc(F)ccc12]

This method of manufacture comprises the steps of:
1) reacting 6-fluoroindole with an iminium ion species generated in-situ from formaldehyde and dimethylamine in the presence of an acidic aqueous solution to produce Compound (II)

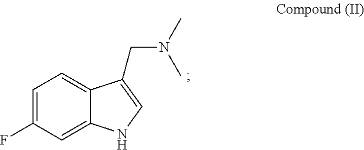

2) reacting Compound(II) with KCN in the presence of DMF-water to produce Compound (III);

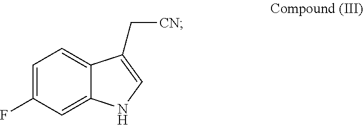 [SMILES: N#CCc1c[nH]c2cc(F)ccc12]

3) hydrogenation of Compound (III) in the presence of NH₃ using Raney nickel (RaNi) to produce Compound (IV);

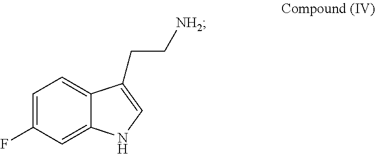 [SMILES: NCCc1c[nH]c2cc(F)ccc12]

4) and reacting Compound (IV) with 3-(2,2,3,3-tetrafluoropropoxy)-benzaldehyde (Compound (IX)) in the presence of a solvent followed by the addition of reducing agent.

The hydrogenation of (6-fluoro-1H-indol-3-yl)acetonitrile (Compound (III)) to 2-(6-fluoro-1H-indol-3-yl)ethylamine (Compound (IV)) disclosed in WO2011/076212 comprises more specifically the steps of:
(a) mixing (6-fluoro-1H-indol-3-yl)acetonitrile, aq. ammonia and a RaNi catalyst in an alcoholic solvent; and
(b) hydrogenating the mixture with H₂.

The synthesis of Compound (IX) can conveniently be carried out as illustrated in Scheme B.

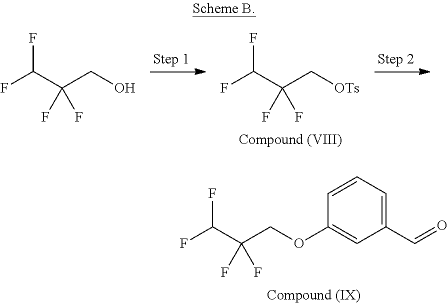 [SMILES: OCC(F)(F)C(F)F ; Cc1ccc(S(=O)(=O)OCC(F)(F)C(F)F)cc1 ; O=Cc1cccc(OCC(F)(F)C(F)F)c1]

The synthesis of Compound (IX) comprises the following steps:
1) Subjecting 2,2,3,3-tetrafluoro-1-propanol to tosylation to yield Compound (VIII);
2) and reacting Compound (VIII) in a displacement reaction with 3-hydroxybenzaldehyde in the presence of a base to yield Compound (IX).

WO2011/076212 further discloses 2-(6-fluoro-1H-indol-3-yl)ethylamine hydrogen L-(+)-tartrate (the 1:1 salt of 2-(6-fluoro-1H-indol-3-yl)ethylamine and L-(+)-tartaric acid) as well as a process for the purification of 2-(6-fluoro-1H-indol-3-yl)ethylamine comprising the steps of:

(a) dissolving 2-(6-fluoro-1H-indol-3-yl)ethylamine in methanol;

(b) adding a solution of L-(+)-tartaric acid in methanol; and

(c) filtering off the tartaric acid salt precipitate.

The use of Raney Nickel in an industrial production is, however, problematic as it easily ignites if it becomes dry during storage, use or as waste. Hence, an alternative cost effective and selective method for the synthesis of N-(2-(6-fluoro-1H-indol-3-yl)-ethyl)-3-(2,2,3,3-tetrafluoropropoxy)-benzylamine is desirable, which avoids the use of Raney Nickel without any significant loss in yield. Such a method has been found and is disclosed in this patent application.

A synthetic route for the starting material 6-fluoroindole (Compound (X)) is via the classical Leimgruber-Batcho indole synthesis. However, as previously reported (Gillmore, A. T. et al., Org. Proc. Res. Dev. 2012, 16, 1897-1904; Boini, S. et al., Org. Proc. Res. Dev. 2006, 10, 1205-1211), isolation and handling of the enamine intermediate, e.g. Compound (XII), is often problematic due to thermal instability. Therefore, a modified Leimgruber-Batcho indole synthesis has been developed and is disclosed herein.

### SUMMARY OF THE INVENTION

In one embodiment of the invention is disclosed a process for the preparation of Compound (IV)

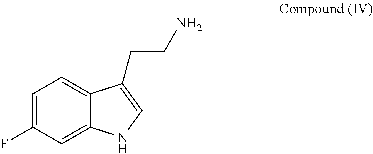 [SMILES: NCCc1c[nH]c2cc(F)ccc12]

comprising the steps of:
(a) mixing Compound (III), (6-fluoro-1H-indol-3-yl)acetonitrile, NH₃ in water and a supported nickel catalyst in a solvent; and
(b) hydrogenating the mixture with hydrogen.

In another embodiment of the invention is disclosed a process for the preparation of Compound (I)

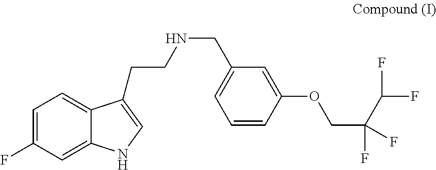 [SMILES: Fc1ccc2c(CCNCc3cccc(OCC(F)(F)C(F)F)c3)c[nH]c2c1]

comprising the above mentioned steps of the process for the preparation of Compound (IV).

In another embodiment of the invention is disclosed a process for the preparation of Compound (X)

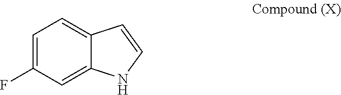

via a modified Leimgruber-Batcho indole synthesis. This new synthetic route of Compound (X) avoids the need to isolate Compound (XII) as illustrated in Scheme C:

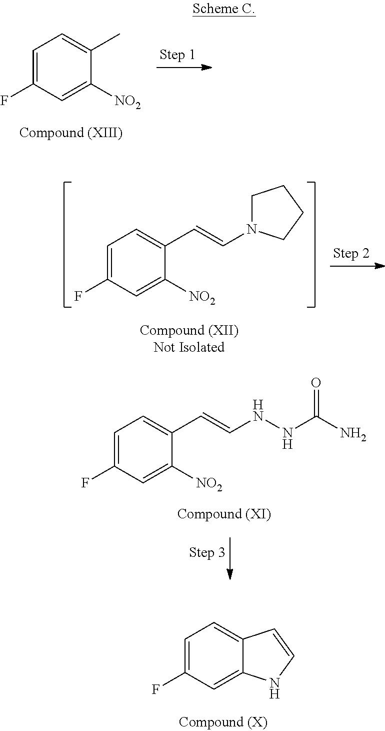

The synthesis of Compound (X) comprises the following steps:
(a) reacting Compound (XIII) with pyrrolidine and an acetal of DMF in a solvent, and subsequently treating the obtained mixture with semicarbazide hydrochloride to obtain solid Compound (XI),
(b) subjecting Compound (XI) to a reduction step with a catalyst and a reductant to yield Compound (X).

### DETAILED DESCRIPTION OF THE INVENTION

The following are definitions for various abbreviations as used throughout the description and claims:

“DEM” is diethoxymethane.

### “DMF” is N,N-dimethylformamide.

“MeOH” is methanol.
“THF” is tetrahydrofuran.
“TCE” is 2,2,2-trichloroethanol.
“i-PrOH” is 2-propanol (isopropyl alcohol).
“OTs” is p-toluensulfonate
“RaNi”/“Raney nickel” is an activated nickel catalyst which is optionally doped with another metal and that comes in different particle sizes and forms
“Cyanide source” is KCN, NaCN, or other agents which release the CN⁻ anion.
“aq” is aqueous.
“DI” is distilled or ultra-pure.
“rt” is room temperature.
“approx.” is approximately
“min” is minutes
“h” is hours
“eq” is equivalents.
“g” is grams.
“mL” is milliliter.
“L” is liter.
“kg” is kilogram.
“M” is molar.
“w/w” is weight per weight.
“v/v” is volume per volume.
“HPLC” is high pressure liquid chromatography.
“LC-MS” is liquid chromatography-mass spectrometry
“Pd/C” is palladium on charcoal.
“Pt/C” is platinum on charcoal.
“Rh/C” is rhodium on charcoal.
“Rh/Alumina” is rhodium on aluminium oxide.
“Ni/Silica-alumina” is nickel on a mixture of silicon oxide and aluminium oxide
“PRICAT™” is the trademark for a series of supported nickel catalysts on silica with/without added promotors, from Johnson Matthey Process Technologies.

### “NMP” is N-methylpyrrolidinone.

“DMF-DMA” is N,N-dimethylformamide dimethyl acetal.
“EDG” is ethylene glycol.

Throughout the description and claims the term “nickel catalyst” refers to catalysts comprising nickel or nickel oxides or mixtures thereof.

In one embodiment of the invention is disclosed a process for the preparation of Compound (IV)

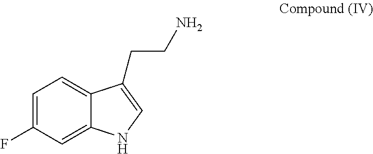 [SMILES: NCCc1c[nH]c2cc(F)ccc12]

comprising the steps of:
(a) mixing (6-fluoro-1H-indol-3-yl)acetonitrile, NH₃ in water and a supported nickel catalyst in a solvent; and
(b) hydrogenating the mixture with hydrogen.

In a first particular embodiment the solvent is an alcoholic solvent.

In a second particular embodiment the nickel catalyst is supported on silica or alumina.

In a third particular embodiment of any of the preceeding embodiments the supported nickel catalyst is selected from the group comprising PRICAT 55/5P and PRICAT 62/15P.

In a fourth particular embodiment of any of the preceeding embodiments the alcoholic solvent is methanol, ethanol or 2-propanol.

In a fifth particular embodiment of any of the preceeding embodiments the hydrogenation is run at a pressure from approx. 2 to approx. 10 bar, more particularly from approx. 2 to approx. 6 bar and most particularly from approx. 2 to approx. 4 bar.

In a sixth particular embodiment of any of the preceeding embodiments the hydrogenation is run at a temperature from about 40° C. to about 70° C., more particularly from about 50° C. to about 60° C.

In a seventh particular embodiment of any of the preceeding embodiments the hydrogenation is run with a loading from about 8% to about 31% (w/w) supported nickel catalyst relative to (6-fluoro-1H-indol-3-yl)acetonitrile.

In another embodiment of the invention is disclosed a process for the preparation of Compound (I)

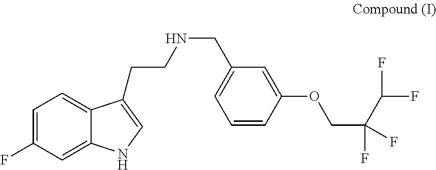 [SMILES: Fc1ccc2c(CCNCc3cccc(OCC(F)(F)C(F)F)c3)c[nH]c2c1]

comprising the steps of any of the above mentioned embodiments of the process for the preparation of Compound (IV).

In a particular embodiment Compound (IV) is reacted with Compound (IX) in a solvent followed by reduction to give yield Compound (I).

In a more particular embodiment sodium borohydride is used as the reducing agent for the reduction to Compound I.

In another embodiment of the invention is disclosed a process for the preparation of Compound (X)

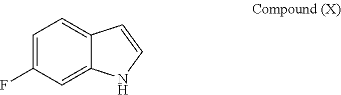

comprising the steps of:
(c) reacting Compound (XIII) with pyrrolidine and an acetal of DMF in a solvent, and subsequently treating the obtained mixture with semicarbazide hydrochloride to obtain solid Compound (XI),
(d) subjecting Compound (XI) to a reduction step with a catalyst and a reductant to yield Compound (X).

In a particular embodiment Compound (XIII) is reacted in DMF or NMP as solvent.

In a more particular embodiment Compound (XIII) is converted to Compound (XI) using DMF-DMA.

In a particular embodiment Compound (XI) is reduced to Compound (X) using Raney nickel or palladium on charcoal as catalyst.

In a more particular Compound (XI) is reduced to Compound (X) using hydrazine or hydrogen as reductant.

Compound (I) forms pharmaceutically acceptable acid addition salts with a wide variety of organic and inorganic acids and include the physiologically acceptable salts which are often used in pharmaceutical chemistry. Such salts are also part of this invention. Such salts include the pharmaceutically acceptable salts listed in Berge, S. M. et al., J. Pharm. Sci. 1977, 66, 1-19 which are known to the skilled artisan. Typical inorganic acids used to form such salts include hydrochloric, hydrobromic, hydriodic, nitric, sulfuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include chloride, bromide, iodide, nitrate, acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, isobutyrate, phenylbutyrate, a-hydroxybutyrate, butyne-1,4-dicarboxylate, hexyne-1,4-dicarboxylate, caprate, caprylate, cinnamate, citrate, formate, fumarate, glycollate, heptarioate, hippurate, lactate, malate, maleate, hydroxymaleate, malonate, mandelate, mesylate, nicotinate, isonicotinate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartrate, and the like.

### EXPERIMENTAL SECTION

### General Experimental

Unless otherwise stated, all reactions were carried out under nitrogen. Reactions were monitored by LC-MS. All reagents were purchased and used without further purification. NMR spectra were recorded at 500 or 600 MHz (¹H NMR), and calibrated to the residual solvent peak. The following abbreviations are used for NMR data: s, singlet; d, doublet; t, triplet; m, multiplet. Coupling constants are rounded to nearest 0.5 Hz.

LC-MS Method:

Acquity UPLC BEH C18 1.7 μm column; 2.1×50 mm operating at 60° C. with flow 1.2 mL/min of a binary gradient consisting of water+0.1% formic acid (A) and acetonitrile+5% water+0.1% formic acid (B). UV detection at 254 nm.

HPLC Method:

Xterra RP18 column (100 mm×4.6 mm, 3.5 μm), mobile phase: 10 mM Ammonium carbonate (pH 8.5)/Acetonitrile, 86/14 to 14/86 (v/v, %), flow rate: 2 mL/min, column temperature: about 45° C., detection: UV at 280 nm.

Compound List:
(I): N-(2-(6-Fluoro-1H-indol-3-yl)-ethyl-(2,2,3,3-tetrafluoropropoxy)-benzylamine
(II): (6-Fluoro-1H-indol-3-ylmethyl)-dimethylamine
(III): 2-(6-Fluoro-1H-indol-3-yl)acetonitrile
(IV): 2-(6-Fluoro-1H-indol-3-yl)ethylamine
(V): 2-(6-Fluoro-1H-indol-3-yl)ethylamine hydrogen L-(+)-tartrate
(VI): 2-(1H-Indol-3-yl)ethylamine
(VII): Bis(2-(6-Fluoro-1H-indol-3-yl)ethyl)amine
(VIII): 2,2,3,3-Tetrafluoropropyl p-toluenesulfonate
(IX): 3-(2,2,3,3-Tetrafluoropropoxy)benzaldehyde
(X): 6-Fluoroindole
(XI): (E)-2-(4-fluoro-2-nitrostyryl)hydrazine-1-carboxamide
(XII): (E)-1-(4-fluoro-2-nitrostyryl)pyrrolidine
(XIII): 4-fluoro-1-methyl-2-nitrobenzene

### Example 1

### Synthesis of Compound (XI)

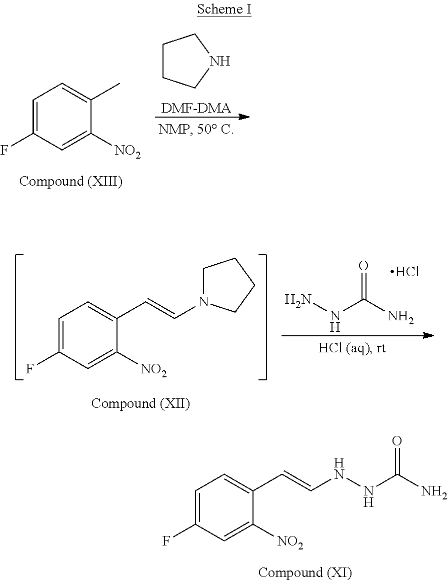

Compound (XIII) (5.0 g, 32.2 mmol) is dissolved in NMP (10 mL). DMF-DMA (4.8 g, 40.3 mmol) and pyrrolidine (3.0 g, 42.0 mmol) is added and the reaction is warmed to 50° C. and stirred for 18 h. The resulting solution is then added to a stirred 50° C. warm solution of semicarbazide hydrochloride (4.7 g, 41.9 mmol) and aq. HCl (36% w/w, 2 mL) in water (40 mL) and stirred for 2 h. The reaction mixture is cooled to 20° C. and the formed orange solid is filtered off, washed with water and dried under vacuum at 50° C. for 18 h to yield Compound (XI) (6.4 g, 83%) with >95% purity according to ¹H NMR analysis.

### Example 2

### Synthesis of Compound (X)

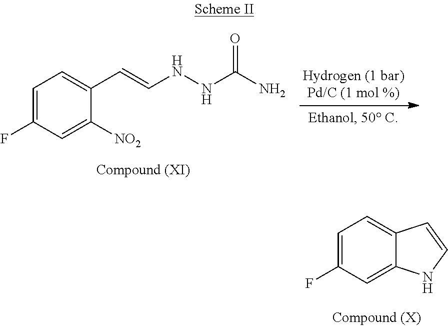 [SMILES: NC(=O)NN/C=C/c1ccc(F)cc1[N+](=O)[O-] ; Fc1ccc2cc[nH]c2c1]

A mixture of Compound (XI) (7.50 g, 31.2 mmol) and palladium on carbon (5% Pd loading, Johnson Matthey type 338, 59.4% w/w water) (1.64 g, 0.312 mmol) in ethanol (75 ml) was hydrogenated at 50° C. and 1.2 bar hydrogen for 3 h.

The reaction mixture was filtered, and the filtrate was evaporated to dryness. The solid residue was heated with ethanol (50 mL) at 50° C. to yield a homogeneous solution. Water (50 mL) was then added dropwise at 50° C. with vigorous stirring. The resulting mixture was concentrated on the rotary evaporator in vacuum at 40° C. to approx. ½ volume. The resulting suspension was filtered, and the precipitate was washed with water and dried in vacuum at 40° C. to Compound (X) (3.58 g, 85%) as an off-white solid, with 100% UV purity according to LC-MS analysis.

### Example 3

### Synthesis of Compound (II)

Details of the synthesis of Compound (II) from commercially available 6-fluoroindole are provided below. The procedure outlined in Scheme III uses diethoxymethane and dimethylamine to generate the “iminium ion species”. An alternative procedure using formaldehyde in place of diethoxymethane is also provided below.

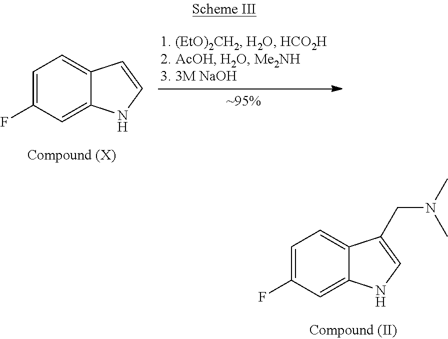

Procedure Using Diethoxymethane

To reactor A were charged diethoxymethane (DEM) (65 mL, 0.52 mol), water (50 mL) and formic acid (39 mL, 1.02 mol)). The mixture was heated at approx. 80° C. (reflux) for approx. 2 h and then cooled to approx. 20° C. To reactor B were charged 6-fluoroindole (50 g, 0.37 mol) and 80% acetic acid (66 mL, 1.17 mol). The suspension was cooled to 2-5° C. 40% Aq. dimethylamine (103 mL, 2.04 mol) was added dropwise to reactor B keeping the temperature below approx. 15° C. The reaction mixture was stirred for approx. 20 min and at the same time the temperature was adjusted to 2-4° C.

The mixture from reactor A (DEM, water, formic acid, formaldehyde and ethanol at about 20° C.) was added drop-wise to reactor B while keeping the temperature at 2-8° C. The reaction mixture was stirred for additional 10 min at 2-8° C. The reaction mixture was slowly warmed to approx. 40° C. over a 1 h period. The reaction mixture was stirred at approx. 40° C. for an additional 1 h. The reaction mixture was cooled to about 20° C.

To reactor C was charged aq. NaOH (800 mL, 2.40 mol, 3 M) and the solution was cooled to about 10° C. The reaction mixture from reactor B was added dropwise to the NaOH solution in reactor C while keeping the temperature at 10-15° C. (pH>14). The suspension was stirred for 40 min at 5-20° C. (pH>14). The product was collected by filtration and the filter-cake was washed twice with water (2×250 mL). The product was dried at approx. 60° C. under vacuum for 16 h to yield Compound (II) (67.6 g, 95%) with 98% UV purity in HPLC analysis.

Procedure Using Formaldehyde:

A 250 L reactor was charged with approx. 40% aq. dimethylamine (35.7 kg, 317 mol) at approx. 17° C. under an inert atmosphere. The mixture was cooled to approx. 4.5° C. and glacial acetic acid (43.4 kg, 723 mol) was added dropwise over 140 min while maintaining the temperature at approx. 15° C. After stirring for 20 min at about 3° C., 37% aqueous formaldehyde (25.9 kg, 319 mol) was slowly added over about 20 min while keeping the temperature between approx. 0° C. to approx. 10° C. 6-Fluoroindole (39.2 kg, 290 mol) was added. The reaction was exothermic and reached a final temperature of approx. 40° C., and it was then cooled down to approx. 20° C. The reaction solution was slowly added to a 650 L reactor previously charged with aq. NaOH (3 M) over a period of approx. 40 min. The formed suspension was stirred for approx. 40 min while keeping the temperature between 5 to 20° C. The precipitate was filtered from solution, washed with water on the filter, and dried at approx. 50° C. to afford Compound (II) (45.4 kg, 81%).

### Example 4

### Synthesis of Compound (III)

A detailed synthesis of Compound (III) from Compound (II) is provided below in Scheme IV.

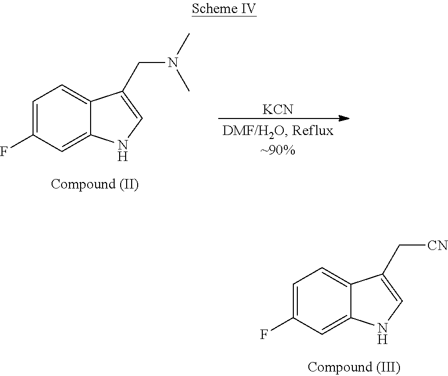

Step-Wise Procedure:

(6-Fluoro-1H-indol-3-ylmethyl)-dimethylamine (II) (65 g, 0.338 mol), KCN (31 g, 0.476 mol), DMF (195 mL) and water (104 mL) were charged to the reactor. The reaction mixture was heated to about 100-105° C. (strong reflux) for about 5-8 h. The reaction mixture was cooled to 20-25° C. Water (780 mL) and toluene (435 mL) were charged to the reactor and the mixture was stirred vigorously for >2 h. The organic and aqueous layers were separated. The organic layer was washed with 5% NaHCO₃ (6×260 mL), aq. HCl (260 mL, 2 M), 5% NaHCO₃ (260 mL) and 5% NaCl (260 mL), respectively. The organic layer was filtered and concentrated to dryness. MeOH (260 mL) was added and the solution was concentrated to dryness to yield Compound (III) as a brown oil (53.0 g, 90%) with 95% UV purity according to HPLC analysis.

### Example 5

### Screening of Palladium Catalysts

To a solution of Compound (III) (200 mg, 1.15 mmol) in EtOH (2.0 mL) was added additive and Pd/C catalyst at rt. The mixture was hydrogenated at 4 bar at the specified temperature for the specified time. The reaction mixture was analysed directly by LC-MS. The results are listed in Table 1.

TABLE 1
Screening of heterogeneous palladium catalysts¹.

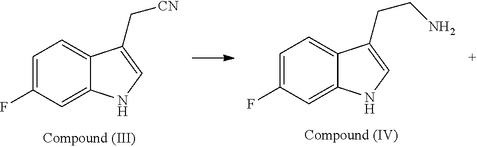 [SMILES: N#CCc1c[nH]c2cc(F)ccc12 ; NCCc1c[nH]c2cc(F)ccc12]

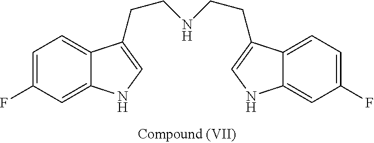 [SMILES: Fc1ccc2c(CCNCCc3c[nH]c4cc(F)ccc34)c[nH]c2c1]

| Pd/C catalyst² | % Cat.⁴ | Temp./° C. | Time/h | Additive | III/%³ | VII/%³ | IV/%³ |
|---|---|---|---|---|---|---|---|
| A102023-5 | 5 | 70 | 20 | 5 eq. CHCl₃ | 5 | 9 | 70 |
| A102023-5 | 5 | 70 | 2.5 | 5 eq. H₂SO₄ | 0 | 28 | 56 |
| A102023-5 | 5 | 60 | 20 | 5 eq. NH₃ in MeOH (7M) | 0 | 22 | 2 |
| A102023-5 | 2 | 70 | 24 | 1 eq. Aq. HCl (12M) | 10 | 14 | 49 |
| A102023-5 | 2 | 50 | 24 | 5 eq. CHCl₃ | 28 | 10 | 53 |
| A102023-5 | 2 | 70 | 48 | None | 2 | 20 | 1 |
| A102023-5 | 2 | 70 | 1 | MsOH | 0 | 30 | 31 |
| A102023-5 | 2 | 100 | 48 | 5 eq. CHCl₃ | 0 | 44 | 8 |
| A102023-5 | 2 | 70 | 48 | 5 eq. Aq. HCl (12M) | 10 | 13 | 36 |
| A102023-5 | 2 | 70 | 48 | Ac₂O | ND | ND | 32 |
| A102023-5 | 2 | 70 | 1 | 5 eq. CHCl₃, 5 eq. DMF- | 43 | 8 | 38 |
| | | | | DMA | | | |
| A102023-5 | 2 | 70 | 48 | Boc₂O | 0 | ND | 24 |
| A102023-5 | 2 | 70 | 48 | 5 eq. HCl | ND | ND | 10 |
| A102023-5 | 2 | 70 | 64 | 5 eq. CHCl₃ | 25 | 18 | 39 |
| A102023-5 | 5 | 70 | 19 | 5 eq. CHCl₃ | 7 | 10 | 64 |
| A102023-5 | 5 | 70 | 19 | 5 eq. C₂Cl₆ | 2 | 2 | 30 |
| A102023-5 | 5 | 70 | 19 | 5 eq. Cl₃CCH₃ | 2 | 2 | 45 |
| A102023-5 | 5 | 70 | 19 | 5 eq. TCE | 2 | 5 | 48 |
| 331 | 2 | 70 | 24 | 5 eq. CHCl₃ | 62 | 8 | 21 |
| 331 | 2 | 50 | 24 | 5 eq. CHCl₃ | 60 | 6 | 26 |
| 331 | 2 | 70 | 64 | 5 eq. CHCl₃ | 60 | 9 | 19 |
| 338 | 2 | 70 | 24 | 5 eq. CHCl₃ | 56 | 11 | 20 |
| 338 | 2 | 50 | 24 | 5 eq. CHCl₃ | 59 | 9 | 26 |
| 338 | 2 | 70 | 64 | 5 eq. CHCl₃ | 74 | 6 | 11 |
| 394 | 5 | 70 | 20 | 5 eq. CHCl₃ | 14 | 14 | 54 |
| 394 | 5 | 70 | 2.5 | 5 eq. H₂SO₄ | 0 | 20 | 51 |
| 394 | 5 | 60 | 20 | 5 eq. NH₃ in MeOH (7M) | 0 | 35 | 3 |
| A503038-5 | 2 | 70 | 24 | 5 eq. CHCl₃ | 38 | 13 | 35 |
| A503038-5 | 2 | 50 | 24 | 5 eq. CHCl₃ | 33 | 10 | 50 |
| 39 | 2 | 70 | 64 | 5 eq. CHCl₃ | 52 | 10 | 26 |
| 39 | 5 | 70 | 19 | 5 eq. CHCl₃ | 5 | 14 | 61 |
| 39 | 5 | 70 | 19 | 5 eq. C₂Cl₆ | 2 | 2 | 21 |
| 39 | 5 | 70 | 19 | 5 eq. Cl₃CCH₃ | 5 | 13 | 52 |
| 39 | 5 | 70 | 19 | 5 eq. TCE | 5 | 13 | 48 |
| 38H | 2 | 70 | 64 | 5 eq. CHCl₃ | 41 | 14 | 30 |
| 87L | 2 | 70 | 64 | 5 eq. CHCl₃ | 11 | 51 | 23 |
| 424 | 2 | 70 | 64 | 5 eq. CHCl₃ | 46 | 13 | 27 |
| 440 | 2 | 70 | 64 | 5 eq. CHCl₃ | 36 | 15 | 22 |

¹Reaction conditions according to the general method.
²Catalysts obtained from Johnson Matthey Process Technology.
³UV-area percentage in LC-MS.
⁴Loading of catalyst in mol % catalyst relative to Compound (III).

### Example 6

### Screening of Homogenous Catalysts

To a solid mixture of metal complex and any ligand was added solvent (1.0 mL). The mixture was stirred for 30 min, and added to a mixture of additive (10 mol %) and Compound (III) (200 mg, 1.15 mmol) in solvent (1.0 mL).

The mixture was hydrogenated at 4 bar and at the specified temperature for the specified time. The reaction mixture was analysed directly by LC-MS.

TABLE 2
Screening of homogeneous catalysts¹

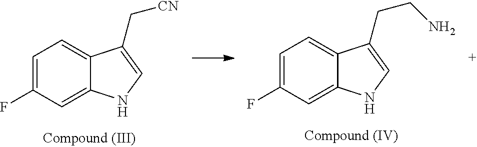 [SMILES: N#CCc1c[nH]c2cc(F)ccc12 ; NCCc1c[nH]c2cc(F)ccc12]

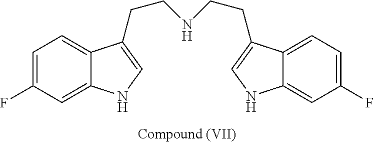 [SMILES: Fc1ccc2c(CCNCCc3c[nH]c4cc(F)ccc34)c[nH]c2c1]

| | | | | | | | | VII/ | IV/ |
|---|---|---|---|---|---|---|---|---|---|
| Metal complex | Ligand | % Cat.⁹ | Temp/° C. | Time/h | Additive | Solvent | III/%² | %² | %² |
| [(Me-allyl)(COD)Ru]₂³ | DPPF⁴ | 1 | 110 | 16 | KOtBu | PhMe | 25 | 0 | 49 |
| [(Me-allyl)(COD)Ru]₂³ | — | 1 | 110 | 16 | KOtBu | PhMe | 84 | 0 | 0 |
| (PPh₃)₃RuCl₂⁵ | — | 1 | 110 | 16 | KOtBu | PhMe | 75 | 0 | 4 |
| (PPh₃)₃RhH(CO)⁶ | — | 1 | 110 | 16 | KOtBu | PhMe | 78 | 0 | 0 |
| (MesRuCl)₂⁷ | PPh₃ | 1 | 110 | 16 | KOtBu | PhMe | 79 | 0 | 0 |
| [(p-cymene)RuCl₂]₂⁸ | DPPB⁹ | 2 | 120 | 2.5 | NaOH | 2- | 72 | 0 | 11 |
| | | | | | | butanol | | | |

¹Reaction conditions according to the general method.
²UV-area percentage in LC-MS.
³Bis(2-methylallyl)(1,5-cyclooctadiene)ruthenium(II), cas number 12289-94-0.
⁴DPPF: 1,1′-Bis(diphenylphosphino)ferrocene, cas number: 12150-46-8.
⁵Tris(triphenylphosphine)ruthenium(II) dichloride, cas number 15529-49-4.
⁶Tris(triphenylphosphine)rhodium(I) carbonyl hydride, cas number 17185-29-4.
⁷Dichloro(mesitylene)ruthenium(II) dimer, cas number 52462-31-4.
⁸Dichloro(p-cymene)ruthenium(II) dimer, cas number 52462-29-0.
⁹Loading of catalyst in mol % catalyst relative to Compound (III).

### Example 7

### Screening of Rhodium, Platinum and Nickel Catalysts

To a solution of Compound (III) (200 mg, 1.15 mmol) in solvent was added additive and catalyst at rt. The mixture was hydrogenated at 4 bar at the specified temperature for the specified time. The reaction mixture was analysed directly by LC-MS.

TABLE 3
Screening of rhodium and platinum catalysts¹

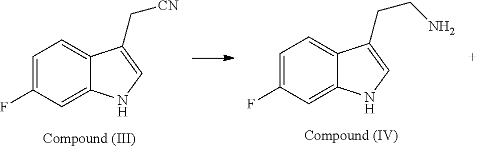 [SMILES: N#CCc1c[nH]c2cc(F)ccc12 ; NCCc1c[nH]c2cc(F)ccc12]

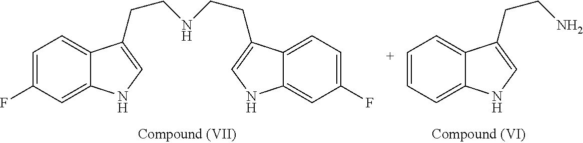

| | | Temp/° | Time/ | | Sol- | | VII/ | | |
|---|---|---|---|---|---|---|---|---|---|
| Catalyst² | % Cat⁴ | C. | h | Additive | vent | III/%³ | %³ | VI/%³ | IV/%³ |
| Rh/C (JM 20A) | 1 | rt | 25 | Aq. NH₃ (32%) | — | 24 | 8 | 0 | 45 |
| | | | | (29 eq. NH₃) | | | | | |
| Rh/C (JM 20A) | 1 | 40 | 25 | Aq. NH₃ (32%) | — | 21 | 4 | 0 | 63 |
| | | | | (29 eq. NH₃) | | | | | |
| Rh/C (JM 20A) | 1 | 60 | 25 | Aq. NH₃ (32%) | — | 0 | 4 | 0 | 82 |
| | | | | (29 eq. NH₃) | | | | | |
| Rh/C (JM 20A) | 1 | 60 | 2.1 | NH₃ (7M) (12 | MeOH | 0 | 17 | 0 | 71 |
| | | | | eq. NH₃) | | | | | |
| Rh/C (JM 20A) | 1 | 40 | 4 | NH₃ (7M) (12 | MeOH | 0 | 21 | 0 | 69 |
| | | | | eq. NH₃) | | | | | |
| Rh/C (JM 20A) | 1 | rt | 4 | NH₃ (7M) (12 | MeOH | 0 | 24 | 0 | 67 |
| | | | | eq. NH₃) | | | | | |
| Rh/C (JM 20A) | 1 | rt | 22 | 5 eq. MsOH | EtOH | 0 | 6 | 0 | 69 |
| Rh/C (JM 20A) | 1 | rt | 22 | 5 eq. H₂SO₄ | EtOH | 0 | 8 | 0 | 51 |
| Rh/C (JM 20A) | 0.4 | 68 | 22 | 83% v/v aq. | EtOH | 0 | 8 | 0 | 60 |
| | | | | NH₃ (32%) (24 | | | | | |
| | | | | eq. NH₃) | | | | | |
| Rh/C (JM 20A) | 0.4 | 60 | 15 | 10 mol % LiOH | EtOH | 0 | 30 | 1 | 65 |
| Rh/C (JM 20A) | 0.4 | 60 | 20 | 40% v/v aq. | EtOH | 0 | 10 | <5 | 61 |
| | | | | NH₃ (32%) (12 | | | | | |
| | | | | eq. NH₃) | | | | | |
| Rh/C (JM 20A) | 0.4 | 70 | 20 | 40% v/v aq. | EtOH | 0 | 11 | <5 | 57 |
| | | | | NH₃ (32%) (12 | | | | | |
| | | | | eq. NH₃) | | | | | |
| Rh/C (JM 20A) | 0.4 | 50 | 20 | 40% v/v aq. | MeOH | 0 | 17 | 0 | 72 |
| | | | | NH₃ (32%) (12 | | | | | |
| | | | | eq. NH₃) | | | | | |
| Rh/C (JM 20A) | 0.4 | 60 | 20 | 40% v/v aq. | MeOH | 0 | 12 | <5 | 59 |
| | | | | NH₃ (32%) (12 | | | | | |
| | | | | eq. NH₃) | | | | | |
| Rh/C (JM 20A) | 0.4 | 60 | 20 | 40% v/v aq. | IPA | 0 | 11 | 0 | 73 |
| | | | | NH₃ (32%) (12 | | | | | |
| | | | | eq. NH₃) | | | | | |
| Rh/C (JM 20A) | 0.4 | 70 | 20 | 40% v/v aq. | IPA | 0 | 11 | 0 | 68 |
| | | | | NH₃ (32%) (12 | | | | | |
| | | | | eq. NH₃) | | | | | |
| Rh/C | 1 | 60 | 10 | 40% v/v aq. | EtOH | 0 | 13 | 0 | 70 |
| (JM C101023-5) | | | | NH₃ (32%) (12 | | | | | |
| | | | | eq. NH₃) | | | | | |
| Rh/Alumina | 1 | 60 | 24 | 40% v/v aq. | EtOH | 0 | 16 | 0 | 80 |
| (JM 526) | | | | NH₃ (32%) (12 | | | | | |
| | | | | eq. NH₃) | | | | | |
| Rh/Alumina | 1 | 60 | 24 | 40% v/v aq. | EtOH | 0 | 17 | 0 | 79 |
| (JM C301011-5) | | | | NH₃ (32%) (12 | | | | | |
| | | | | eq. NH₃) | | | | | |
| Rh/Alumina | 1 | 60 | 24 | 40% v/v aq. | EtOH | 0 | 16 | 0 | 82 |
| (JM 524) | | | | NH₃ (32%) (12 | | | | | |
| | | | | eq. NH₃) | | | | | |
| Rh/Alumina | 1 | 60 | 10 | 61% v/v aq. | MeOH | 0 | 24 | 0 | 70 |
| (JM 524) | | | | NH₃ (32%) (15 | | | | | |
| | | | | eq. NH₃) | | | | | |
| Rh/Alumina | 1 | 60 | 10 | 67% v/v aq. | MeOH | 0 | 17 | 0 | 79 |
| (JM 524) | | | | NH₃ (32%) (27 | | | | | |
| | | | | eq. NH₃) | | | | | |
| Rh/Alumina | 1 | 60 | 10 | 68% v/v aq. | MeOH | 0 | 15 | 0 | 79 |
| (JM 524) | | | | NH₃ (32%) (35 | | | | | |
| | | | | eq. NH₃) | | | | | |
| Rh/Alumina | 1 | 50 | 24 | 68% v/v aq. | MeOH | 0 | 17 | 0 | 79 |
| (JM 524) | | | | NH₃ (32%) (35 | | | | | |
| | | | | eq. NH₃) | | | | | |
| Rh/Alumina | 1 | 60 | 10 | 68% v/v aq. | EtOH | 0 | 11 | 0 | 75 |
| (JM 524) | | | | NH₃ (32%) (35 | | | | | |
| | | | | eq. NH₃) | | | | | |
| Rh/Alumina | 0.5 | 60 | 24 | 68% v/v aq. | MeOH | 0 | 19 | 0 | 56 |
| (JM 524) | | | | NH₃ (32%) (35 | | | | | |
| | | | | eq. NH₃) | | | | | |
| Rh/Alumina | 0.25 | 60 | 36 | 68% v/v aq. | MeOH | 0 | 17 | 0 | 38 |
| (JM 524) | | | | NH₃ (32%) (35 | | | | | |
| | | | | eq. NH₃) | | | | | |
| Rh/Alumina | 1 | 60 | 10 | 74% v/v aq. | IPA | 0 | 15 | 0 | 80 |
| (JM 524) | | | | NH₃ (32%) (30 | | | | | |
| | | | | eq. NH₃) | | | | | |
| Rh/Alumina | 1 | 60 | 10 | 77% v/v aq. | IPA | 0 | 13 | 0 | 84 |
| (JM 524) | | | | NH₃ (32%) (44 | | | | | |
| | | | | eq. NH₃) | | | | | |
| Rh/Alumina | 1 | 70 | 10 | 77% v/v aq. | IPA | 0 | 10 | 0 | 78 |
| (JM 524) | | | | NH₃ (32%) (44 | | | | | |
| | | | | eq. NH₃) | | | | | |
| Rh/Alumina | 1 | 80 | 10 | 77% v/v aq. | IPA | 3 | 8 | 0 | 64 |
| (JM 524) | | | | NH₃ (32%) (44 | | | | | |
| | | | | eq. NH₃) | | | | | |
| Rh/Alumina | 1 | 90 | 8 | 77% v/v aq. | IPA | 2 | 7 | 0 | 71 |
| (JM 524) | | | | NH₃ (32%) (44 | | | | | |
| | | | | eq. NH₃) | | | | | |
| Rh/Alumina | 0.5 | 80 | 15 | 77% v/v aq. | IPA | 15 | 4 | 0 | 61 |
| (JM 524) | | | | NH₃ (32%) (44 | | | | | |
| | | | | eq. NH₃) | | | | | |
| Rh/Alumina | 0.25 | 80 | 19 | 77% v/v aq. | IPA | 39 | 2 | 0 | 39 |
| (JM 524) | | | | NH₃ (32%) (44 | | | | | |
| | | | | eq. NH₃) | | | | | |
| Rh/Alumina | 1 | 60 | 10 | 77% v/v aq. | IPA | 0 | 11 | 0 | 85 |
| (JM 524) | | | | NH₃ (32%) (44 | | | | | |
| | | | | eq. NH₃) | | | | | |
| Rh/Alumina | 1 | 60 | 10 | 77% v/v aq. | EtOH | 0 | 8 | 0 | 73 |
| (JM 524) | | | | NH₃ (32%) (44 | | | | | |
| | | | | eq. NH₃) | | | | | |
| Rh/Alumina | 2 | 60 | 6 | 77% v/v aq. | IPA | 0 | 12 | 0 | 79 |
| (JM 524) | | | | NH₃ (32%) (44 | | | | | |
| | | | | eq. NH₃) | | | | | |
| Rh/Alumina | 3 | 60 | 5 | 77% v/v aq. | IPA | 0 | <1 | 0 | 82 |
| (JM 524) | | | | NH₃ (32%) (44 | | | | | |
| | | | | eq. NH₃) | | | | | |
| Rh/Alumina | 0.5 | 60 | 21 | 77% v/v aq. | IPA | 11 | 7 | 0 | 74 |
| (JM 524) | | | | NH₃ (32%) (44 | | | | | |
| | | | | eq. NH₃) | | | | | |
| Rh/Alumina | 5 | rt | 14 | 50% v/v aq. | EtOH | 0 | 21 | 10 | 69 |
| (S-A) | | | | NH₃ (32%) (15 | | | | | |
| | | | | eq. NH₃) | | | | | |
| Rh/Alumina | 5 | 40 | 14 | 50% v/v aq. | EtOH | 0 | 17 | 7 | 73 |
| (S-A) | | | | NH₃ (32%) (15 | | | | | |
| | | | | eq. NH₃) | | | | | |
| Rh/Alumina | 5 | 60 | 14 | 50% v/v aq. | EtOH | 0 | 10 | 26 | 64 |
| (S-A) | | | | NH₃ (32%) (15 | | | | | |
| | | | | eq. NH₃) | | | | | |
| Rh/Alumina | 1 | 60 | 14 | 40% v/v aq. | EtOH | 0 | 11 | 0 | 85 |
| (S-A) | | | | NH₃ (32%) (12 | | | | | |
| | | | | eq. NH₃) | | | | | |
| Rh/Alumina | 1 | 70 | 14 | 40% v/v aq. | EtOH | 0 | 11 | 0 | 84 |
| (S-A) | | | | NH₃ (32%) (12 | | | | | |
| | | | | eq. NH₃) | | | | | |
| Rh/Alumina | 1 | 60 | 24 | 40% v/v aq. | IPA | 0 | 9 | 0 | 83 |
| (S-A) | | | | NH₃ (32%) (12 | | | | | |
| | | | | eq. NH₃) | | | | | |
| Rh/Alumina | 1 | 60 | 24 | 40% v/v aq. | MeOH | 0 | 13 | <5 | 66 |
| (S-A) | | | | NH₃ (32%) (12 | | | | | |
| | | | | eq. NH₃) | | | | | |
| Rh/Alumina | 1 | 60 | 24 | 40% v/v aq. | EtOH | 0 | 9 | <5 | 71 |
| (S-A) | | | | NH₃ (32%) (12 | | | | | |
| | | | | eq. NH₃) | | | | | |
| Rh/Alumina | 1 | 60 | 24 | 40% v/v aq. | EDG | 0 | 11 | 0 | 75 |
| (S-A) | | | | NH₃ (32%) (12 | | | | | |
| | | | | eq. NH₃) | | | | | |
| Rh/Alumina | 1 | 60 | 10 | 77% v/v aq. | IPA | 0 | 9 | 0 | 88 |
| (S-A) | | | | NH₃ (32%) (44 | | | | | |
| | | | | eq. NH₃) | | | | | |
| Pt/C (JM 117) | 1 | 60 | 24 | 40% v/v aq. | EtOH | 62 | 14 | <5 | 11 |
| | | | | NH₃ (32%) | | | | | |

Reaction conditions according to the general method.
¹Catalysts obtained from Johnson Matthey Process Technology (designation: JM) or Sigma-Aldrich A/S (designation: S-A).
²UV-area percentage in LC-MS.
³Loading of catalyst in mol % catalyst relative to Compound (III).

TABLE 4
Screening of Nickel catalysts¹

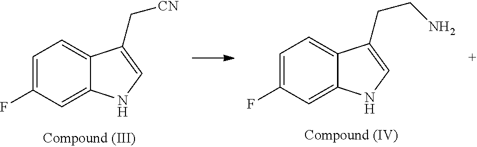 [SMILES: N#CCc1c[nH]c2cc(F)ccc12 ; NCCc1c[nH]c2cc(F)ccc12]

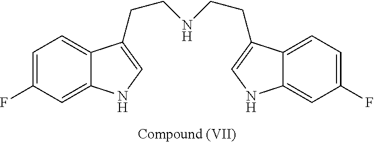 [SMILES: Fc1ccc2c(CCNCCc3c[nH]c4cc(F)ccc34)c[nH]c2c1]

| | % Cat | Temp/° | Time/ | | | | VII/ | |
|---|---|---|---|---|---|---|---|---|
| Catalyst² | (w/w) | C. | h | Additive | Solvent | III/%³ | %³ | IV/%³ |
| Ni/silica- | 11 | 60 | 10 | 40% v/v aq. NH₃ | EtOH | 46 | <4 | 37 |
| alumina | | | | (32%) (12 eq. NH₃) | | | | |
| (Aldrich) | | | | | | | | |
| PRICAT | 12 | 60 | 10 | 40% v/v aq. NH₃ | EtOH | 29 | <2 | 49 |
| 55/5P | | | | (32%) (12 eq. NH₃) | | | | |
| PRICAT | 12 | 60 | 10 | 40% v/v aq. NH₃ | EtOH | 50 | 3 | 36 |
| 62/15P | | | | (32%) (12 eq. NH₃) | | | | |
| PRICAT | 29 | 50 | 20 | 79% v/v aq. NH₃ | EtOH | 0 | <0.6 | 91 |
| 62/15P | | | | (32%) (44 eq. NH₃) | | | | |
| PRICAT | 31 | 50 | 21 | 40% v/v aq. NH₃ | EtOH | 0 | 13 | 81 |
| 55/5P | | | | (32%) (12 eq. NH₃) | | | | |
| PRICAT | 31 | 50 | 9 | 79% v/v aq. NH₃ | EtOH | 0 | <0.6 | 96 |
| 55/5P | | | | (32%) (44 eq. NH₃) | | | | |
| PRICAT | 31 | 60 | 6 | 79% v/v aq. NH₃ | EtOH | 0 | <0.6 | 92 |
| 55/5P | | | | (32%) (44 eq. NH₃) | | | | |
| PRICAT | 31 | 50 | 8 | 79% v/v aq. NH₃ | IPA | 0 | <0.6 | 95 |
| 55/5P | | | | (32%) (44 eq. NH₃) | | | | |
| PRICAT | 31 | 50 | 9.5 | 79% v/v aq. NH₃ | MeOH | 0 | <0.6 | 94 |
| 55/5P | | | | (32%) (44 eq. NH₃) | | | | |
| PRICAT | 31 | 50 | 20 | 79% v/v aq. NH₃ | EtOH | 0 | <0.6 | 91 |
| 55/5P | | | | (32%) (44 eq. NH₃) | | | | |
| PRICAT | 31 | 50 | 10 | 65% v/v aq. NH₃ | IPA | 0 | <0.6 | 94 |
| 55/5P | | | | (32%) (15 eq. NH₃) | | | | |
| PRICAT | 31 | 50 | 10 | 67% v/v aq. NH₃ | IPA | 0 | <0.6 | 93 |
| 55/5P | | | | (32%) (23 eq. NH₃) | | | | |
| PRICAT | 31 | 50 | 10 | 75% v/v aq. NH₃ | IPA | 0 | <0.6 | 95 |
| 55/5P | | | | (32%) (35 eq. NH₃) | | | | |
| PRICAT | 24 | 50 | 15 | 65% v/v aq. NH₃ | IPA | 0 | <0.6 | 93 |
| 55/5P | | | | (32%) (15 eq. NH₃) | | | | |
| PRICAT | 24 | 50 | 15 | 67% v/v aq. NH₃ | IPA | 0 | <0.6 | 93 |
| 55/5P | | | | (32%) (23 eq. NH₃) | | | | |
| PRICAT | 24 | 50 | 15 | 75% v/v aq. NH₃ | IPA | 0 | <0.6 | 95 |
| 55/5P | | | | (32%) (35 eq. NH₃) | | | | |
| PRICAT | 24 | 50 | 12 | 65% v/v aq. NH₃ | IPA | 0 | <0.6 | 92 |
| 55/5P | | | | (32%) (15 eq. NH₃) | | | | |
| PRICAT | 20 | 50 | 17 | 65% v/v aq. NH₃ | IPA | 0 | <0.6 | 86 |
| 55/5P | | | | (32%) (15 eq. NH₃) | | | | |
| PRICAT | 16 | 50 | 20 | 65% v/v aq. NH₃ | IPA | 0 | <0.6 | 88 |
| 55/5P | | | | (32%) (15 eq. NH₃) | | | | |
| PRICAT | 12 | 50 | 30 | 65% v/v aq. NH₃ | IPA | 0 | <0.6 | 89 |
| 55/5P | | | | (32%) (15 eq. NH₃) | | | | |
| PRICAT | 8 | 50 | 40 | 65% v/v aq. NH₃ | IPA | 0 | <0.6 | 81 |
| 55/5P | | | | (32%) (15 eq. NH₃) | | | | |

¹Reaction conditions according to the general method.
²Catalysts obtained from Johnson Matthey Process Technology, except for the first which was obtained from Sigma-Aldrich.
³UV-area percentage in LC-MS.
⁴Loading of catalyst in weight % catalyst relative to Compound (III).

### Example 8

### Synthesis of 2-(6-Fluoro-1H-indol-3-yl)ethylamine hydrogen L-(+)-tartrate (V)

To a solution of Compound (III) (10.0 g, 57.4 mmol, 96% UV purity in LC-MS) in aqueous ammonia (59.2 g, 65.0 mL, 834 mmol, 24% w/w) and IPA (35.0 mL) was added PRICAT type 55/5P catalyst (3.0 g) at rt. The mixture was transferred to a steel autoclave and hydrogenated at 4 bar hydrogen for 23 h at 50° C. The mixture was cooled and filtered through a glass microfibre filter (Whatman GF/A) using additional IPA (35 mL). The filtrate was concentrated by evaporation in vacuo to approx. ⅓ volume. IPA (70 mL) was added, and the mixture was again concentrated to approx. ⅓ volume. The IPA addition and evaporation sequence was repeated twice. The last time the mixture was evaporated to dryness in vacuo.

The residue was dissolved in IPA (200 mL) and water (10 mL) was added. The solution was heated to reflux. Then a solution of L-(+)-Tartaric acid (8.62 g, 57.4 mmol) in water (30 mL) was slowly added over a period of 10 min to the stirred solution at reflux. The resulting solution was slowly cooled to rt with stirring. The formed suspension was filtered and the precipitate was washed with cold IPA (50 mL) and dried in vacuo to yield Compound (V) (14.5 g, 77% yield) as a white powder with >99.9% UV purity in LC-MS analysis.

Analytical data for Compound (V): ¹H NMR (600 MHz, CDCl₃) δ_{H} 2.96 (t, J=7.5 Hz, 2H), 3.05 (t, J=7.5 Hz, 2H), 6.87 (dt, J=2.0, 10 Hz, 1H), 7.14 (dd, J=2.0, 10 Hz, 1H), 7.54 (dd, J=5.5, 10.0 Hz, 1H), 11.1 (br s, 1H); ¹³C NMR (150 MHz, DMSO-d₆) δ_{C} 23.6, 39.7, 72.4 (tartrate), 97.9 (d, J=25.5 Hz), 107.4 (d, J=24.5 Hz), 110.4, 119.6 (d, J=10.0 Hz), 124.0, 124.5, 136.6 (d, J=12.5 Hz), 159.4 (d, J=232.5 Hz), 175.2 (tartrate); LC-MS (APPI): m/e calc. for C₁₀H₁₂FN₂ [M+H]⁺ 179.10. found 179.2 (free base).

### Example 9

### Large Scale Synthesis of 2-(6-fluoro-1H-indol-3-yl)ethylamine hydrogen L-tartrate (V)

Hydrogenation

PRICAT type 55/5P catalyst (14.0 kg) was charged to a reactor followed by charging of a solution of Compound (III) (46.3 kg, 266 mol) in isopropanol (76.4 kg). Then isopropanol (106 L) and aq. ammonia (302 L, 25%) was charged. The mixture was transferred to a steel autoclave under nitrogen, using extra isopropanol (92 L) for washing of reactor. The autoclave was evacuated and then pressurized with hydrogen gas to 3 bar. The content was heated to 55° C. and hydrogenated at 3 bar hydrogen for 48 h. The content was cooled to 25° C., and the autoclave was purged with nitrogen gas, and the content filtered on a pressure nutsch filter. The filter was washed with isopropanol (2×145 L). This yielded a solution of Compound (IV).

Precipitation

The amount of solution of Compound (IV) from two hydrogenations of the above size was concentrated by vacuum destillation to smallest possible volume, diluted with IPA (486 L) and again concentrated by vacuum destillation. This was repeated twice with two batches of isopropanol (285 L and then 306 L). Then isopropanol (930 L) and ethyl acetate (450 kg) was added, and the mixture was heated to 60° C. A solution of L-(+)-tartaric acid (39.9 kg, 26.6 mol) in water (85 L) and isopropanol (280 L) was added slowly over a period of approx. 30 min to the solution. The formed suspension was stirred at 60° C. for 3 h, and cooled over a period of 3 h to 25° C. The suspension was filtered on a pressure nutsch filter, and the filter cake was washed twice with a mixture of isopropanol (170 L), ethyl acetate (78 kg) and water (17 L). The filter cake was broken up and dried on trays in a vacuum oven at 60° C. for 5 days to yield Compound (V) (163 kg, 94%) as an off-white solid.

### Example 10

### Precipitation of 2-(6-fluoro-1H-indol-3-yl)ethylamine hydrogen L-(+)-tartrate (V)

Compound (IV) (5.4 g, 30.3 mmol) was dissolved in isopropyl alcohol (60 mL) and was heated to 60° C. A solution of L-(+)-tartaric acid (4.55 g, 30.3 mmol) in water (12 mL) was prepared, and approx. one third of this solution was added dropwise over 5 min, and the solution was allowed to stir for a further 10 min prior to seeding. Precipitation was observed. A further one third of this solution was added dropwise, and after 10 min the remainder of the aqueous solution was added dropwise. The suspension was allowed to stir at 60° C. for 30 min, and then was allowed to cool to 50° C., and was stirred at that temperature for 1 h. The suspension was then allowed to cool to room temperature (approx. 22° C.) overnight (approx. 16 h). The suspension was filtered, and the residue was dried under vacuum to give Compound (V) (7.7 g, 77% yield) as a solid.

### Example 11

### Precipitation of 2-(6-fluoro-1H-indol-3-yl)ethylamine hydrogen L-(+)-tartrate (I) from crude 2-(6-fluoro-1H-indol-3-yl)ethyl-1-amine (III) after hydrogenation

Crude Compound (IV) (329 g, 1.8 mol) was dissolved in isopropanol (660 mL) and the solution was warmed to 50° C. This was transferred to a 10 L flask, and more isopropanol (2.3 L) was added. The resultant solution was then heated to and maintained at 60° C. using a thermostatically-controlled heating mantle. Separately, a solution of L-(+)-tartaric acid (246 g, 1.6 mol) in water (650 mL) was prepared, total volume 800 mL. A portion of this aqueous solution (266 mL) was added to the solution of the amine at a rate of 25 mL/min. After approximately 80 mL of the solution was added, precipitation was observed. A further 130 mL of the solution was added at a rate of 2 mL/min. The remainder of the solution was then added at a rate of 6 mL/min. The heating mantle was then turned off, and the suspension was allowed to cool overnight to 23° C. (approx. 17 h). The suspension was then cooled to 20° C. using a water bath, and filtered. The filter cake was broken up and dried under vacuum at 50° C. to give Compound (V) (443 g, 73%) as a solid.

### Example 12

### Synthesis of Compound (IX)

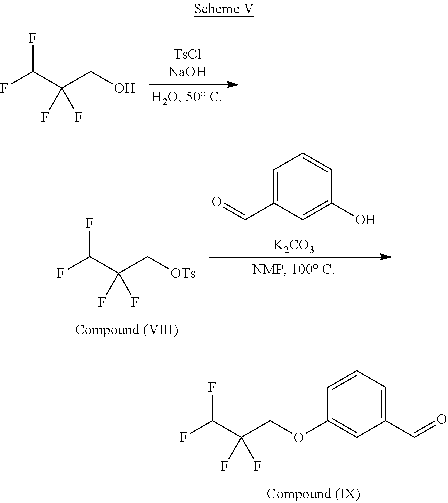

To p-toluenesulfonyl chloride (140 g, 0.734 mol) was added 2,2,3,3-tetrafluoro-1-propanol (100 g, 0.757 mol) followed by water (440 mL). The mixture was stirred while aq. NaOH (100 mL, 27.7% w/w) was added slowly. The mixture was heated to 50° C. and maintained at that temperature for 5 h. The mixture was cooled to rt, and toluene (700 mL) was added. The mixture was stirred for 15 min, and the phases were separated. The organic phase was washed with aq. ammonia (250 mL, 5% w/w), brine (200 mL, 5% w/w) twice and finally filtered and evaporated to dryness to yield Compound (IX) (183 g, 87%) as a colorless oil.

Crude Compound (VIII) (45.8 g, 0.160 mol) from above was mixed with potassium carbonate (32.2 g, 0.233 mol) and 3-hydroxybenzaldehyde (25.0 g, 0.205 mol) in N-methylpyrrolidinone (137 mL). The mixture was stirred at 90° C. for 1 h, and then at 100° C. for 3 h. The mixture was cooled to 50° C., and water (220 mL) was added. The resulting mixture was added to a mixture of toluene (400 mL), brine (75 mL, 15% w/w), water (200 mL) and aq. NaOH (60 mL, 27.7% w/w). The mixture was stirred briefly and the phases were separated. The organic phase was washed sequentially with aq. NaOH (230 mL, 2 M) twice, aq. HCl (150 mL, 2M), aq. NaHCO₃ (150 mL, 5% w/w), and lastly with brine (50 mL, 5% w/w). The organic phase was filtered and evaporated to dryness in vacuo. The resulting oil was stripped twice with isopropanol (100 mL) to yield Compound (IX) (34.4 g, 91%) as an oil.

### Example 13

### Synthesis of Compound (I) as HCl-Salt

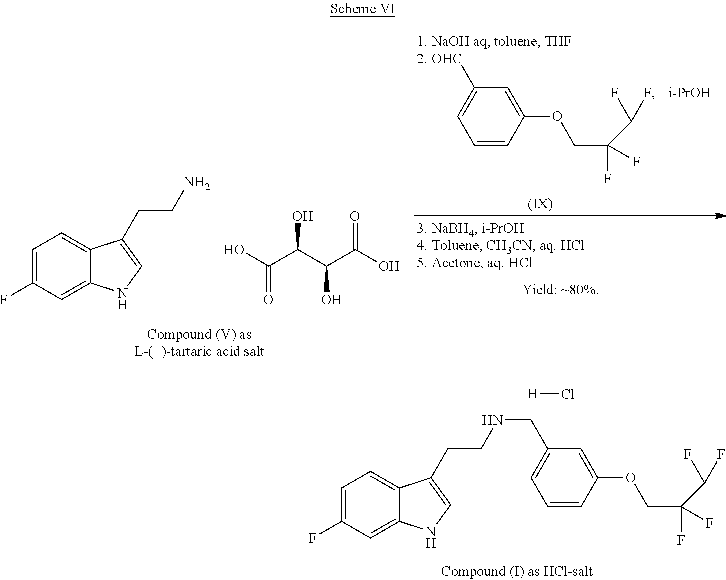

Procedure:

Compound (V) (49.3 g, 0.150 mol) was stirred in a mixture of toluene (270 mL), THF (100 mL), aq. NaOH (200 mL, 2 M) and aq. NaCl (65 mL, 15% w/w). The phases were separated. The organic phase was washed with aq. NaCl (200 mL, 5% w/w). The organic phase was concentrated under reduced pressure to dryness and the residue dissolved in isopropanol (400 mL).

Compound (IX) (39.0 g, 0.165 mol) and isopropanol (200 mL) were charged to the reaction mixture. The reaction mixture was heated at 60° C. for 2.5 h and then cooled to about 55° C. To the hot reaction mixture was charged a suspension of NaBH₄ (7.4 g, 0.196 mol) in isopropanol (100 and 50 mL). The reaction mixture was heated at 55° C. for 2.5 h and then cooled to about 15-20° C. Aq. HCl (80 mL, 2 M) was added dropwise over a period of about 30 min. Aq. HCl (140 mL, 2 M) was added over a period of 15 min. The mixture was stirred vigorously for 15 min. The mixture was concentrated to half volume followed by addition of aq. NaOH (83 mL, 6 M) to pH≧14. Toluene (400 mL) was added. The phases were separated and the organic phase was washed with aq. NaOH (200 mL, 2 M), aq. NH₄Cl (200 mL, 3% w/w) and water (200 mL), respectively. The organic phase was filtered and concentrated to dryness. The residue was dissolved in toluene (550 mL) and acetonitrile (50 mL). Aq. HCl (33 mL, 6 M) was added drop-wise. The resulting suspension was stirred for 2-4 hours and then filtered. The filter-cake was washed with toluene:acetonitrile mixture (9:1, 2×75 mL) and aq. HCl (2×75 mL, 0.1 M), respectively. The crude HCl salt of Compound (I) was dried under vacuum at about 45° C. for about 16 h.

Final purification of the HCl salt of Compound (I) was performed by first dissolving the isolated salt in acetone (300 mL). The solution was filtered and concentrated to a volume of about 90-120 mL. Filtered aq. HCl (1900 mL, 0.1 M) was added dropwise over 30 min. The resulting suspension was stirred at 20-25° C. for 16 h and then filtered. The filtercake was washed with filtered HCl (200 mL, 0.1 M) and filtered water (150 mL), respectively. The purified HCl salt of Compound (I) (52.2 g, 80%) was dried at 40° C. under vacuum for about 16 h and isolated as a white solid with >99.5% UV purity in HPLC analysis.

1. A process for the preparation of Compound (IV) 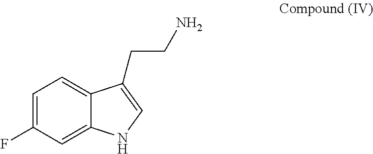 [SMILES: NCCc1c[nH]c2cc(F)ccc12]
comprising the steps of:
(a) mixing (6-fluoro-1H-indol-3-yl)acetonitrile, NH₃ in water and a supported nickel catalyst in a solvent; and
(b) hydrogenating the mixture with H₂.

2. The process of claim 1, wherein the solvent is an alcoholic solvent.

3. The process of claim 1, wherein the nickel catalyst is supported by silica or alumina.

4. The process of claim 1, wherein the supported nickel catalyst is selected from the group comprising PRICAT 55/5P and PRICAT 62/15P.

5. The process of claim 1, wherein the alcoholic solvent is methanol, ethanol or 2-propanol.

6. The process of claim 1, wherein the hydrogenation is run at a pressure of from about 2 to about 10 bars.

7. The process of claim 1, wherein the hydrogenation is run at a temperature from about 40° C. to about 70° C.

8. The process of claim 1, wherein the hydrogenation is run with a loading from about 8% to about 31% (w/w) supported nickel catalyst relative to (6-fluoro-1H-indol-3-yl)acetonitrile.

9. A process for the preparation of Compound (X) 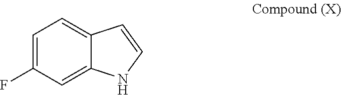
comprising the steps of:
a. reacting 4-fluoro-1-methyl-2-nitrobenzene with pyrrolidine and an acetal of N,N-dimethylformamide in a solvent, and subsequently treating the obtained mixture with semicarbazide hydrochloride to obtain solid (E)-2-(4-fluoro-2-nitrostyryl)hydrazine-1-carboxamide, and
b. subjecting (E)-2-(4-fluoro-2-nitrostyryl)hydrazine-1-carboxamide to a reduction step with a catalyst and a reductant to yield Compound (X).

10. The process according to claim 9, wherein the solvent is N,N-dimethylformamide or N-methylpyrrolidinone.

11. The process according to claim 9, wherein the acetal of N,N-dimethylformamide is N,N-dimethylformamide dimethyl acetal.

12. The process according to claim 9, wherein the catalyst is Raney nickel or palladium on charcoal.

13. The process according to claim 9, wherein the reductant is hydrazine or hydrogen.

14. A process for the preparation of Compound (I) 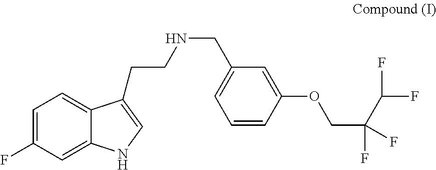 [SMILES: Fc1ccc2c(CCNCc3cccc(OCC(F)(F)C(F)F)c3)c[nH]c2c1]
comprising one or more steps of claim 1.
